(19) United States
(12) Patent Application Publication
(10) Pub. No.: US 2013/0157990 A1
(43) Pub. Date: Jun. 20, 2013

(54) METHOD OF DRUG DETOXIFICATION

(71) Applicant: EAGLE ADVANCEMENT INSTITUTE LLC, West Bloomfield, MI (US)

(72) Inventors: James W. Carpenter, Lake Orion, MI (US); Ricardo D. Borrego, Grosse Pointe Farms, MI (US)

(73) Assignee: EAGLE ADVANCEMENT INSTITUTE LLC, West Bloomfield, MI (US)

(21) Appl. No.: 13/771,602

(22) Filed: Feb. 20, 2013

Related U.S. Application Data
(63) Continuation-in-part of application No. PCT/US2011/048593, filed on Aug. 22, 2011.
(60) Provisional application No. 61/375,581, filed on Aug. 20, 2010.

(51) Int. Cl. A61K 31/485; A61K 31/404; A61K 31/194; A61K 31/426; A61K 31/40; A61K 31/4178; A61K 31/551; A61K 31/195; A61K 31/05; A61K 31/407; A61K 31/27; A61K 31/439; A61K 31/166; A61K 31/4168; A61K 31/5415; A61K 31/495; A61K 31/215; A61K 31/451; A61K 31/46; A61K 31/5513; A61K 31/5517; A61K 31/437; A61K 31/573

(52) U.S. Cl. CPC A61K 31/485; A61K 31/573; A61K 31/404; A61K 31/194; A61K 31/426; A61K 31/40; A61K 31/4178; A61K 31/551; A61K 31/195; A61K 31/05; A61K 31/407; A61K 31/27; A61K 31/439; A61K 31/166; A61K 31/4168; A61K 31/5415; A61K 31/495; A61K 31/215; A61K 31/451; A61K 31/46; A61K 31/5513; A61K 31/5517; A61K 31/437
USPC 514/171; 514/418; 514/574; 514/370; 514/424; 514/397; 514/220; 514/561; 514/282; 514/413; 514/538; 514/294; 514/401; 514/226.2; 514/256; 514/529; 514/225.8; 514/327; 514/304; 514/221; 514/300

(57) ABSTRACT

A method of detoxifying a patient addicted to at least one drug comprises the step of administering a first-medication component to the patient for mitigating drug withdrawal symptoms of the patient. The method further comprises the step of administering an anesthetic component to the patient for putting the patient in a sedated state. The method further comprises the step of administering a detoxification component to the patient for detoxifying the patient of the drug(s) while the patient is in the sedated state. The detoxification component can comprise naloxone. The method further comprises the step of administering a second-medication component to the patient for further mitigating drug withdrawal symptoms of the patient while the patient is in the sedated state. The second-medication component can comprise naltrexone. The method further comprises the step of terminating administration of the anesthetic component to the patient for reviving the patient from the sedated state.

### CROSS REFERENCE TO RELATED APPLICATIONS

This application is a continuation-in-part (C-I-P) of International Application Serial No. PCT/US2011/048593, with an international filing date of Aug. 22, 2011, which claims the benefit of U.S. Provisional Patent Application Ser. No. 61/375,581, with a U.S. filing date of Aug. 20, 2010, both of which are incorporated herein by reference in their entirety.

### FIELD OF THE INVENTION

The present invention generally relates to a method of drug detoxification and more specifically to a method of opiate detoxification.

### DESCRIPTION OF THE RELATED ART

Drug addiction is a state of periodic or chronic intoxication produced by the repeated consumption of a drug. Characteristics of drug addiction include: compulsion to continue taking the drug and to obtain it by any means, tendency to increase dosage of the drug, physical and/or psychological dependence on the drug, etc.

Addiction occurs when continued use of the drug alters the chemical composition of the brain. The drug induces the brain to release the endorphins dopamine and serotonin, natural pleasure causing substances within the reward circuit of the brain, which cause a drug induced euphoria. Immediately after a high, a protein activated in the brain cuts off dopamine release and temporarily inhibits the reward circuit. In chronic drug users, a sustained activation of this protein forces a larger dose of the drug for endorphin release, thereby increasing the body's dependence on the drug.

Opiate withdrawal symptoms are the greatest obstacle most people experience when trying to quit use of a drug. Some of these withdrawal symptoms include: nausea, vomiting, diarrhea, tremors, muscle aches, hot and cold flashes, anxiety, and/or agitation. While not generally life-threatening, these symptoms prevent many people from breaking the cycle of drug addiction, creating a permanent environment for continued dependence.

Various methods and programs have been developed to address drug addiction and withdrawal symptoms, such as rapid detoxification procedures. Many of these methods suffer from one or more deficiencies, such as complexity, patient suffering, side effects, cost, and/or short and long term effectiveness.

Many programs for drug detoxification offer to assist an addicted individual through an initial physical process of eliminating drugs from their bodies, while attempting to mitigate immediate and cumbersome withdrawal symptoms, which typically inhibit an addict from drug cessation on their own. Generally, patients are sedated, detoxified, monitored for short periods of time, and sent home with recommendations for follow up support they must seek on their own. There are no continuing physical or psychological support measures, including medications for residual withdrawal symptoms, which can continue to occur randomly and periodically for months afterward.

Of the various conventional methods for abstaining from drug use, unfortunately the average success rate after one year is very low (e.g. less than 20%). If someone chooses to go “cold turkey”, i.e., stop drug use on their own volition, the average success rate is less than 5% after one year. The same is true for basic outpatient procedures. Simply put, less than 1 in 20 former drug abusers/users remain drug free after a year if relying on such “basic” methods for treatment. While more “moderate” methods such as intensive outpatient procedures and residential type programs generally offer better results, success rates for these methods still only range from 10% to 20% after one year. Simply put, less than 1 in 5, if not less than 1 in 10, former drug abusers/users remain drug free after a year if relying on even more moderate methods for treatment. Some of these methods (typically the moderate methods) use certain types of drugs in an attempt to wean an abuser off of another drug (e.g. Vicodin, opiates, etc.); however, this can lead to a new dependency on the weaning drug itself (e.g. Methadone or Suboxone).

In view of the foregoing, there remains an opportunity to provide improved methods for drug detoxification. Specifically, there remains an opportunity to provide a method of rapid drug detoxification and treatment which has excellent success rates relative to conventional treatment methods.

### SUMMARY OF THE INVENTION AND ADVANTAGES

A method of detoxifying a patient addicted to at least one drug comprises the step of administering a first-medication component to the patient for mitigating drug withdrawal symptoms of the patient. The first-medication component generally comprises gabapentin, ropinirole, citric acid sodium citrate, famotidine, glycopyrrolate, ondansetron, dexamethasone, midazolam, or combinations thereof. The method further comprises the step of administering an anesthetic component to the patient for putting the patient in a sedated state. The method further comprises the step of administering a detoxification component to the patient for detoxifying the patient of the drug(s) while the patient is in the sedated state. The method further comprises the step of administering a second-medication component to the patient for further mitigating drug withdrawal symptoms of the patient while the patient is in the sedated state. The method further comprises the step of terminating administration of the anesthetic component to the patient for reviving the patient from the sedated state.

The method of the present invention provides excellent detoxification results for the patient. The method of the present invention also generally reduces patient pain and discomfort associated with conventional detoxification methods. The patient of the method of the present invention is also less likely to relapse relative to conventional detoxification methods. Said another way, the invention method generally has excellent success rates by attacking both the physical and psychological addictions/dependencies of the patient which is useful for preventing relapse of the patient over time, such as after one year or more.

### DETAILED DESCRIPTION OF THE INVENTION

The present invention provides a method of detoxifying a patient addicted to at least one drug. The invention method is also useful for preventing relapse of the patient over time. It is to be appreciated that the patient may be addicted to one or a plurality of different drugs. Typically, the drug is an opiate. For example, the drug can be heroin, or any preparation or derivative of opium. As used herein, the term opiate may also refer to an opioid, e.g. a synthetic narcotic. It is to be appreciated that the drug may be different than an opiate. Examples of other drugs include, but are not limited to, Actiq, Alfenta, Alodan, Anexsia, Avinza, Benzodiazepine, Buprenex, Buprenorphine, Butorphanol, Centralgin, Codeine, Darvocet, Darvon, Demerol, Dihydrocodeine, Dilaudid, Dimepheptanol, Dinarkon, Dispadol, Dolantin, Dolargan, Dolestine, Dolophine, Dolosal, Dolsin, Duragesic Patch, Endocet, Endodan, Eukodal, Fentanyl, Fiorinal, Hydromorphone, Hydrocodone, Kadian, levo-α-acetylmethadol (LAAM), Levorphanol, Lorcet, Lortab, Mepergan, Meperidine, Mefedina, Meprodine, Methadone, Methadose, Morphine, MS Contin, Norco, Nubain, Numorpitan, Opana (Oxymorphone), Opium, Oramorph, Oramorph SR, ORLMM, Oxycodone, Oxycontin, OxyIR, OxyFAST, Percocet, Percodan, Palladone, Palladone IR, Palladone SR, Percolone, Petidin, Prodine, Propoxyphene, Roxanol, Roxicet, Roxiprin, Roxicodone, Suboxone, Subutex, Supeudol, Tramadol, Tylenol 3 & 4, Thebaine, Tilidine, Tylox, Ultracet, Ultram, Vicodin, Vicoprofen, and combinations thereof.

The method of the present invention generally enables the patient to successfully circumvent uncomfortable and sometimes painful drug withdrawal symptoms. Specifically, the method generally enables the patient to mitigate or reduce the intensity and/or duration of drug withdrawal symptoms typically associated with conventional detoxification methods. The method of the present invention may also be referred to herein as the procedure, the treatment, or the program.

Typically, the method, or portions thereof, is carried out in a clinically equipped medical/surgical environment by one or more licensed and trained anesthesiologists and/or medical staff, as described below. The method may also be carried out in other locations, and is not limited to any one location in particular. Various other types of personnel may also be used to interact with the patient. During the procedure, the patient may be moved to various locations for treatment, such as by gurney, vehicle, etc.

The method comprises the step of administering a first-medication component to the patient for mitigating drug withdrawal symptoms of the patient. The method further comprises the step of administering an anesthetic component to the patient for putting the patient in a sedated state. The method further comprises the step of administering a detoxification component to the patient for detoxifying the patient of the drug(s) while the patient is in the sedated state. The method further comprises the step of administering a second-medication component to the patient for further mitigating drug withdrawal symptoms of the patient while the patient is in the sedated state. The method yet further comprises the step of terminating administration of the anesthetic component to the patient for reviving the patient from the sedated state. The method may also include one or more additional (or optional) steps, as described below.

Optionally, prior to the administering the first-medication component to the patient, information on the patient can be gathered. This information is typically gathered some time prior to administering the first-medication component to the patient, such as a few hours to a day or more beforehand. During this period of time, a patient file and/or chart can be created. Labels for the patient and various components can also be prepared. The patient can also review and sign an informed consent form.

Various types of information on the patient can be gathered. This information can include, but is not limited to, general medical status and/or history of the patient; detailed medical status and/or history of the patient, such as major illnesses, surgeries/operations, current and/or chronic illnesses, childhood illnesses, illnesses in the patient's family, etc.; family or personal issues with anesthesia; identification and demographics of the patient, such as name, age, height, and weight of the patient; social history of the patient, such as tobacco and alcohol use; drug history of the patient, such as regular and acute medication use, over the counter drug use, prescribed or herbal drug use and/or therapy, and history of opiate use, both prescribed and recreational; and the patient's insurance information.

Optionally, to avoid the unnecessary use of an airway tube during the method, the patient can prepare his/her body for the method. As understood in the art, airway tubes are conventionally used for the possibility of aspiration of vomitus. Typically, at least one day prior to the administering the first-medication component to the patient, the patient is instructed to only ingest clear fluids, i.e., no solid foods. Various fluids can be ingested by the patient during this period. Various medications may also be taken by the patient, such as Dulcolax® and Miralax®.

Optionally, prior to the administering the first-medication component to the patient, post procedure instructions can be reviewed and given to the patient and/or the patient's caretaker(s), e.g. family member(s), friend(s), etc. The instructions are useful for care of the patient after the procedure. The procedure itself can also be explained to the patient and/or the patient's caretaker(s).

Typically, prior to the administering the first-medication component to the patient, the patient is examined, e.g. physically and/or psychologically. The patient is typically examined prior to, administering the first-medication component to the patient, such as a few hours to a day or more beforehand. The examination is useful for establishing a base line of the patient.

The examination can include a blood draw and/or an electrocardiography (ECG or “EKG”) of the patient. The patient may also be subjected to a stress test. The patient can also be medically evaluated in other ways understood in the art. Results of this examination can be evaluated. The patient can also be psychologically tested and evaluated in addition to being medically tested and evaluated. Examination and evaluation of the patient can be conducted by various methods, such as by a medical professional, e.g. a physician and/or a psychologist.

Examination of the patient can include a visual inspection, such as inspection for cuts, burns, bruises, sores, wounds, or scabs. Such conditions can be recorded by the medical professional and initialed by the patient to further establish the base line of the patient. During examination, nil per os (NPO) of the patient can be verified. A drug test may be administered to the patient, such as a 7 panel drug test to determine which drugs may be present in the patient. If the patient is a female, a pregnancy test can be administered to the patient, such as a urine β-hCG test, to determine if the patient is pregnant. Results of these tests can evaluated to determine whether or not to proceed further with the treatment at that time.

As introduced above, the first-medication component is administered to the patient. The first-medication component can comprise one or more medications. The first-medication component is useful for mitigating drug withdrawal symptoms of the patient.

The first-medication component can be administered to the patient by various methods understood in the art. For example, the first-medication component can be administered orally (PO), intravenously (IM), or combinations thereof. The specific administration technique can be based in part on the form of the medication(s). For example, these and the other medications utilized herein may be administered to the patient by various administration techniques, such as orally, intravenously, via injection, via inhalation, via transdermal patch, rectally, etc. Typically, at least one intravenous (IV) line is started for purposes of the present invention. For example, the IV line can be used for “pushing” one or more medications into the patient, during administration of the respective medication(s). The first-medication component is typically administered by a medical professional, such as by an anesthesiologist, a paramedic, or a nurse. The first-medication component can be administered in various amounts, as described below.

Typically, the first-medication component comprises, consists essentially of, or consists of, gabapentin, ropinirole, citric acid sodium citrate, famotidine, glycopyrrolate, ondansetron, dexamethasone, midazolam, or combinations thereof. For example, the first-medication component can comprise one or a plurality (i.e., two or more) of the aforementioned medications. In certain embodiments, the first-medication component comprises all of the aforementioned medications. In other embodiments, the first-medication component comprises more than one but less than all of the aforementioned medications. In the various embodiments, each of the aforementioned medications can be used in various amounts.

These medications are available from a variety of sources. Those skilled in the art appreciate that these medications can be provided under a variety of brand names. For example, Neurontin® is a form of gabapentin, Requip® is a form of ropinirole, Bicitra® is a form of citric acid sodium citrate, Pepcid® is a form of famotidine, Robinul® is a form of glycopyrrolate, Zofran® is a form of ondansetron, Decadron® is a form of dexamethasone, and Versed® is a form of midazolam. The medications may also be available generically or under another brand name.

In certain embodiments, the first-medication component comprises gabapentin. The gabapentin can be administered to the patient orally in various amounts, typically in an amount of from about 100 to about 2500, alternatively from about 500 to about 2000, alternatively from about 750 to about 1500, alternatively from about 1000 to about 1500, alternatively about 1200, mg, or any range between the lowest and highest of these values. It is to be appreciated that the gabapentin can be administered in other ways, if desired.

In certain embodiments, the first-medication component comprises ropinirole. The ropinirole can be administered to the patient orally in various amounts, typically in an amount of from about 0.01 to about 2.5, alternatively from about 0.1 to about 1, alternatively from about 0.25 to about 1, alternatively from about 0.25 to about 0.75, alternatively from about 0.25 to about 0.5, alternatively about 0.25, mg, or any range between the lowest and highest of these values. It is to be appreciated that the ropinirole can be administered in other ways, if desired.

In certain embodiments, the first-medication component comprises citric acid sodium citrate. The citric acid sodium citrate can be administered to the patient orally in various amounts, typically in an amount of from about 1 to about 50, alternatively from about 10 to about 40, alternatively from about 15 to about 35, alternatively from about 25 to about 35, alternatively about 30, ml, or any range between the lowest and highest of these values. It is to be appreciated that the citric acid sodium citrate can be administered in other ways, if desired.

In certain embodiments, the first-medication component comprises famotidine. The famotidine can be administered to the patient intravenously (e.g. via piggyback) in various amounts, typically in an amount of from about 1 to about 50, alternatively from about 5 to about 40, alternatively from about 10 to about 40, alternatively from about 10 to about 30, alternatively about 20, mg, or any range between the lowest and highest of these values. It is to be appreciated that the famotidine can be administered in other ways, if desired.

In certain embodiments, the first-medication component comprises glycopyrrolate. The glycopyrrolate can be administered to the patient intravenously in various amounts, typically in an amount of from about 0.01 to about 1, alternatively from about 0.05 to about 1, alternatively from about 0.1 to about 0.5, alternatively from about 0.1 to about 0.25, alternatively about 0.2, mg, or any range between the lowest and highest of these values. It is to be appreciated that the glycopyrrolate can be administered in other ways, if desired.

In certain embodiments, the first-medication component comprises ondansetron. The ondansetron can be administered to the patient intravenously in various amounts, typically in an amount of from about 0.1 to about 10, alternatively from about 1 to about 10, alternatively from about 1 to about 7.5, alternatively from about 1 to about 5, alternatively about 4, mg, or any range between the lowest and highest of these values. It is to be appreciated that the ondansetron can be administered in other ways, if desired.

In certain embodiments, the first-medication component comprises dexamethasone. The dexamethasone can be administered to the patient intravenously in various amounts, typically in an amount of from about 0.1 to about 50, alternatively from about 1 to about 25, alternatively from about 5 to about 20, alternatively from about 5 to about 10, alternatively about 10, mg, or any range between the lowest and highest of these values. It is to be appreciated that the dexamethasone can be administered in other ways, if desired.

In certain embodiments, the first-medication component comprises midazolam. The midazolam can be administered to the patient intravenously in various amounts, typically in an amount of from about 0.01 to about 20, alternatively from about 1 to about 15, alternatively from about 1 to about 10, alternatively from about 1 to about 5, alternatively about 2, mg, or any range between the lowest and highest of these values. It is to be appreciated that the midazolam can be administered in other ways, if desired. It is also to be appreciated that the amounts described above for each of the medications can be used in different combinations with one another, provided the respective medication is included as/in the first-medication component.

Typically, after administering the first-medication component to the patient, the anesthetic component is administered to the patient. The anesthetic component may also be administered prior to or contemporaneous with administration of the first-medication component. The anesthetic component can comprise one or more sedatives. The anesthetic component is useful for putting the patient in a sedated state.

The anesthetic component can be administered to the patient by various methods understood in the art. For example, the anesthetic component can be administered intravenously. The specific administration technique can be based in part on the form of the sedative(s). The anesthetic component is typically administered by a medical professional, such as an anesthesiologist. The anesthetic component can be administered in various amounts, as described below.

Typically, the anesthetic component comprises propofol; however, other sedatives understood in the art may also be used. Other suitable sedatives, for purposes of the present invention, are described in International Publication No. WO 99/63935, U.S. Pat. No. 5,789,411, and U.S. Pat. No. 6,004,962, the disclosures of which are incorporated herein by reference in their entirety to the extent they do not conflict with the general scope of the invention. If any conflict exists between the instant disclosure and any of the incorporated reference(s), only the portion(s) of any of the incorporated reference(s) that are in conflict with the instant disclosure, rather than an entirety of any of the incorporated reference(s), are expunged from incorporation herein from the incorporated reference(s). Propofol and other suitable sedatives are available from a variety of sources. Those skilled in the art appreciate that propofol can be provided under a variety of brand names. For example, Diprivan® is a form of propofol,

In certain embodiments, the anesthetic component comprises propofol. The propofol can be administered to the patient intravenously in various amounts, typically in an amount of from about 0.5 to about 10, alternatively from about 1 to about 1, alternatively from about 2 to about 5, alternatively from about 2.5 to about 4.5, mg per kg (mg/kg), each based on weight of the patient, or any range between the lowest and highest of these values. It is to be appreciated that the anesthetic component can be administered in other ways, if desired.

Typically, a continuous drip of the anesthetic component is administered once the patient is in the sedated state. The continuous drip can be of various rates, typically of from about 50 to about 500, alternatively from about 100 to about 500, alternatively from about 200 to about 400, alternatively about 300, mcg per kg per minute (mcg/kg/min), each based on weight of the patient, or any range between the lowest and highest of these values. The continuous drip can be adjusted to maintain adequate anesthesia (or sedation) of the patient. For example, an anesthesiologist can monitor and adjust the sedation level of the patent over time, as needed.

Typically, one or more monitoring devices are applied to the patient before, after, and/or while the patient is sedated. The monitoring device can be an electrocardiograph (ECG or “EKG”) with one or more leads, a continuous pulse oximeter (SpO2), and/or a blood pressure monitor, such as a cuff. Various types of monitoring devices understood in the art can be utilized. Vitals (or vital signs) of the patient can be taken at various times, such as before, after, and/or during sedation of the patient. Vital signs of the patient can be taken, recorded, and analyzed against a base line of the patient. The vital signs are useful for monitoring the patient, especially while the patient is sedated.

Typically, the patient will be oxygenated with an oxygen mask before the patient is sedated. The patient's airway can also be evaluated. Once an airway has been established, a laryngeal mask airway (LMA) is typically placed over the patient and proper position can be verified. Upon verification of an optimal airway via the LMA, sedation of the patient can commence. The patient is typically ventilated at least while the patient is in the sedated state.

Typically, once general anesthesia of the patient is established, the detoxification component is administered to the patient. The detoxification component can comprise one or more medications. The detoxification component is useful for detoxifying the patient of the drug(s). Specifically, without being bound or limited by any particular theory, it is believed that any active drug(s), e.g. opiate(s), present in the patient's body are cleansed from the body, while the other medications described herein help to mitigate physical withdrawal symptoms that may be experienced by the patient in the absence of such medications.

The detoxification component can be administered to the patient by various methods understood in the art. For example, the detoxification component can be administered intravenously, intramuscularly, or combinations thereof. The specific administration technique can be based in part on the form of the medication(s). The detoxification component is typically administered by a medical professional, such as by an anesthesiologist, a paramedic, or a nurse. The detoxification component can be administered in various amounts, as described below.

Typically, the detoxification component comprises, consists essentially of, or consists of, naloxone, ketorolac, methacarbamol, dolasetron, trimethobenzamide, or combinations thereof. For example, the detoxification component can comprise one or a plurality (i.e., two or more) of the aforementioned medications. Typically, the detoxification component comprises naloxane, and, optionally, at least one of the other medications, which can also be referred to as adjunct medications. In certain embodiments, the detoxification component comprises all of the aforementioned medications. In other embodiments, the detoxification component comprises more than one but less than all of the aforementioned medications. In the various embodiments, each of the aforementioned medications can be used in various amounts.

These medications are available from a variety of sources. Those skilled in the art appreciate that these medications can be provided under a variety of brand names. For example, Narcan® is form of naloxone, Toradol® is a form of ketorolac, Robaxin® is a form of methacarbamol, Anzemet® is a form of dolasetron, and Tigan® is a form of trimethobenzamide. The medications may also be available generically or under another brand name.

In certain embodiments, the detoxification component comprises naloxone. The naloxone can be administered to the patient intravenously in various amounts, typically in an amount of from about 0.01 to about 5, alternatively from about 0.1 to about 5, alternatively from about 1 to about 5, alternatively from about 1 to about 2, alternatively about 1.2, mg, or any range between the lowest and highest of these values. It is to be appreciated that the naloxone can be administered in other ways, if desired.

The naloxone can be made into a solution, such as by combining the naloxone with normal saline, e.g. with 50 ml of normal saline, for purposes of administration. The same is true for other medications described herein, if applicable to the particular medication. As such, the medications described herein can be used in various concentrations.

In certain embodiments, the detoxification component comprises ketorolac. The ketorolac can be administered to the patient intravenously and/or intramuscularly in various amounts, typically in an amount of from about 1 to about 100, alternatively from about 1 to about 75, alternatively from about 10 to about 50, alternatively from about 20 to about 40, alternatively about 30, mg, or any range between the lowest and highest of these values. It is to be appreciated that the ketorolac can be administered in other ways, if desired. In specific embodiments, the ketorolac is administered to the patient intravenously and intramuscularly in a first portion and a second portion. For example, 30 mg of ketorolac can be administered to the patient intravenously and 30 mg of ketorolac can be administered to the patient intramuscularly.

In certain embodiments, the detoxification component comprises methacarbamol. The methacarbamol can be administered to the patient intravenously in various amounts, typically in an amount of from about 1 to about 500, alternatively from about 10 to about 250, alternatively from about 50 to about 150, alternatively from about 75 to about 125, alternatively about 100, mg, or any range between the lowest and highest of these values. It is to be appreciated that the methacarbamol can be administered in other ways, if desired.

In certain embodiments, the detoxification component comprises dolasetron. The dolasetron can be administered to the patient intravenously in various amounts, typically in an amount of from about 0.1 to about 50, alternatively from about 1 to about 25, alternatively from about 5 to about 20, alternatively from about 10 to about 15, alternatively about 12.5, mg, or any range between the lowest and highest of these values. It is to be appreciated that the dolasetron can be administered in other ways, if desired.

In certain embodiments, the detoxification component comprises trimethobenzamide. The trimethobenzamide can be administered to the patient intramuscularly in various amounts, typically in an amount of from about 1 to about 500, alternatively from about 10 to about 250, alternatively from about 50 to about 150, alternatively from about 75 to about 125, alternatively about 100, mg, or any range between the lowest and highest of these values. It is to be appreciated that the trimethobenzamide can be administered in other ways, if desired. It is also to be appreciated that the amounts described above for each of the medications can be used in different combinations with one another, provided the respective medication is included as/in the detoxification component.

Typically, after administering the detoxification component to the patient, the second-medication component is administered to the patient. The second-medication component is useful for further mitigating drug withdrawal symptoms of the patient.

The second-medication component can be administered to the patient by various methods understood in the art. For example, the second-medication component can be administered intravenously, intramuscularly, or combinations thereof. The specific administration technique can be based in part on the form of the medication(s). The second-medication component is typically administered by a medical professional, such as a paramedic or nurse. The second-medication component can be administered in various amounts, as described below.

Typically, the second-medication component comprises, consists essentially of, or consists of, naltrexone, clonidine, or combinations thereof. For example, the second-medication component can comprise one or both of the aforementioned medications. Typically, the second-medication component comprises naltrexone, and, optionally, clonidine. Naltrexone is an opiate antagonist (or blocker). In certain embodiments, the second-medication component comprises both of the aforementioned medications.

These medications are available from a variety of sources. Those skilled in the art appreciate that these medications can be provided under a variety of brand names. For example, Catapres® is a form of clonidine. The medications may also be available generically or under another brand name.

In certain embodiments, the second-medication component comprises naltrexone. The naltrexone can be administered to the patient intravenously and/or intramuscularly. In certain embodiments, the naltrexone is administered to the patient intravenously in various amounts, typically in an amount of from about 0.01 to about 10, alternatively from about 1 to about 10, alternatively from about 1 to about 5, alternatively from about 2 to about 4, alternatively about 3, mg, or any range between the lowest and highest of these values. It is to be appreciated that the naltrexone can be administered in other ways, if desired.

In specific embodiments, the naltrexone is administered to the patient intravenously in a first portion and second portion. For example, two doses of about 1.5 mg of naltrexone can be administered to the patient. The doses can be spaced apart, such as by about 5 minutes.

In certain embodiments, the naltrexone is administered to the patient intramuscularly in various amounts, typically in an amount of from about 1 to about 1000, alternatively from about 100 to about 1000, alternatively from about 250 to about 750, alternatively from about 300 to about 600, alternatively about 500, mg, or any range between the lowest and highest of these values.

In specific embodiments, the naltrexone is administered to the patient intravenously and intramuscularly in a first portion and a second portion. For example, the first portion of naltrexone can be administered to the patient intravenously and the second portion (or remaining portion) of naltrexone can be administered to the patient intramuscularly. The portions can be equal or different from one other. As a further example, two doses of about 1.5 mg of naltrexone can be administered to the patient intravenously. The doses can be spaced apart, such as by about 5 minutes. A dose of about 500 mg of naltrexone can also be administered to the patient intramuscularly. The intramuscular naltrexone can be administered before, after, or simultaneous with administration of the intravenous naltrexone.

In certain embodiments, the second-medication component comprises clonidine. The clonidine can be administered to the patient intramuscularly in various amounts, typically in an amount of from about 0.001 to about 1, alternatively from about 0.01 to about 1, alternatively from about 0.1 to about 1, alternatively from about 0.1 to about 0.5, alternatively about 0.3, mg, or any range between the lowest and highest of these values. It is to be appreciated that the clonidine can be administered in other ways, if desired. It is also to be appreciated that the amounts described above for each of the medications can be used in different combinations with one another, provided the respective medication is included as/in the second-medication component.

In a specific embodiment, the clonidine is administered to the patient intravenously in a first portion and second portion. For example, two doses of 0.15 mg of clonidine can be administered to the patient. The doses can be spaced apart, such as by about 5 minutes.

Typically, after administering the second-medication component to the patient, administration of the anesthetic component to the patient is terminated. Termination of the anesthetic component is useful for reviving the patient from the sedated state. In other words, the anesthetic component is discontinued to initiate emergence of the patient from anesthesia. Typically, after terminating administration of the anesthetic component to the patient, the LMA is removed from the patient. The patient is typically taken out of general anesthesia by a medical professional, such as an anesthesiologist.

Optionally, the patient is examined after terminating administration of the anesthetic component to the patient. The patient is typically examined some time after, such as a few minutes to a few hours or more after, terminating administration of the anesthetic component to the patient. The examination is useful for comparison against the base line of the patient, as described above. The patient is typically examined by a medical professional, such as paramedic or nurse.

Typically, the patient is in the sedated state for a period of time less than about four hours, alternatively less than about three hours, alternatively less than about two hours, alternatively less than about 90 minutes, alternatively less than about 60 minutes, alternatively less than about 45 minutes. As such, the method of the present invention may be referred to in the art as a rapid detoxification method or “rapid detox”. In various embodiments, the invention method may also be referred to as intensive outpatient opiate treatment or “IOOT”. As alluded to above, the sedated state of the patient is generally defined as the period in which the patient is under sedation or general anesthesia. By having a relatively short sedation time, the possibility of the patient suffering from one or more anesthesia related issues, such as vomiting, nausea, sore throat, shivering, and/or delayed normal mental functioning, can be reduced or prevented. The patient is typically no longer considered to be sedated once in an ambulatory state.

As alluded to above, vital signs of the patient can also be monitored after sedation (i.e., once the patient is ambulatory), to observe possible signs of drug withdrawal and/or other complications. Typically, the patient is monitored for at least about 30, alternatively at least about 45, alternatively at least about 60, minutes, after the patient is ambulatory to ensure patient stability. In certain embodiments, the patient is monitored for at least two, alternatively at least three, alternatively at least four, hours, after the patient is ambulatory to ensure stability of the patient. If the patient suffers from drug withdrawal, the patient can be given one or more medications for easing withdrawal symptoms. Suitable medications are described below. Typically, the IV line is removed from the patient once the patient is deemed stable. The monitoring device(s) can also be removed from the patient once the patient is deemed stable.

After the patient is deemed stable, the patient is typically transferred to a recovery location for an observation period. The patient can be observed and cared for by one or more medical professionals, such as a paramedic and/or a nurse. The patient can also be assisted by his/her caregiver, e.g. a family member.

Typically, the patient is observed and attended to for at least 4 hours, alternatively at least about 8, alternatively at least about 12, alternatively at least about 18, alternatively at least about 24, alternatively at least about 36, alternatively at least about 48, alternatively at least about 60, alternatively at least about 72, hours, after the patient is initially deemed stable. The patient can be examined during this period, such as from every about four to about eight hours. The medical professional can be useful for reestablishing an IV line, if needed. The medical professional can also be useful for administering one or more additional medications, as described below. The medical professional is also useful for answering any questions the patient and/or caregiver may have, as well as providing comfort and support for the patient. Once the observation period is over, the patient may be discharged.

As alluded to above, after terminating administration of the anesthetic component to the patient, a third-medication component may be administered to the patient. Typically, the third-medication component is administered to the patient by the medical professional during the observation period of the patient. The caregiver and/or patient may also administer the third-medication component during or after the observation period. The third-medication component is useful for further mitigating drug withdrawal symptoms of the patient. In addition, the third-medication can be useful for preventing or discouraging the patient from taking the drug(s) again. For example, the third-medication component can block effects of the drug(s), thus preventing the patient from becoming easily addicted after detoxification.

The third-medication component can be administered to the patient by various methods understood in the art. For example, the third-medication component can be administered orally, sublingually, intramuscularly, or combinations thereof. The specific administration technique can be based in part on the form of the medication(s). The third-medication component can be administered in various amounts, as described below.

Typically, the third-medication component comprises, consists essentially of, or consists of, clonidine, naltrexone, promethazine, phenobarbital, ropinirole, ondansetron, or combinations thereof. For example, the third-medication component can comprise one or both of the aforementioned medications. Typically, the third-medication component comprises naltrexone, and, optionally, one or more of the other medications. In certain embodiments, the third-medication component comprises all of the aforementioned medications.

These medications are available from a variety of sources. Those skilled in the art appreciate that these medications can be provided under a variety of brand names. For example, Phenergan® is a form of promethazine, and Solfoton® is a form of phenobarbital. The medications may also be available generically or under another brand name.

In certain embodiments, the third-medication component comprises clonidine. The clonidine can be administered to the patient orally in various amounts, typically in an amount of from about 0.01 to about 1, alternatively from about 0.05 to about 1, alternatively from about 0.1 to about 1, alternatively from about 0.1 to about 0.5, alternatively about 0.1, mg, or any range between the lowest and highest of these values. It is to be appreciated that the clonidine can be administered in other ways, if desired. In certain embodiments, the clonidine is administered in one or more doses. For example, the clonidine can be administered in about 0.1 mg doses multiple times, e.g. about 17 times. The doses can be spaced apart, such as by about 8 hours.

In certain embodiments, the third-medication component comprises naltrexone. The naltrexone can be administered to the patient orally, intramuscularly, subcutaneously, or combinations thereof. In certain embodiments, the naltrexone is administered to the patient orally in various amounts, typically in an amount of from about 1 to about 100, alternatively from about 10 to about 90, alternatively from about 25 to about 75, alternatively from about 40 to about 60, alternatively about 50, mg, or any range between the lowest and highest of these values. It is to be appreciated that the naltrexone can be administered in other ways, if desired. In certain embodiments, the naltrexone is administered in one or more doses. For example, the naltrexone can be administered in about 50 mg doses multiple times, e.g. about 30 times. The doses can be spaced apart, such as by about 24 hours.

In certain embodiments, the third-medication component comprises promethazine. The promethazine can be administered to the patient orally in various amounts, typically in an amount of from about 1 to about 50, alternatively from about 5 to about 50, alternatively from about 10 to about 40, alternatively from about 20 to about 30, alternatively about 25, mg, or any range between the lowest and highest of these values. It is to be appreciated that the promethazine can be administered in other ways, if desired. In certain embodiments, the promethazine is administered in one or more doses. For example, the promethazine can be administered in about 25 mg doses multiple times, e.g. about 12 times. The doses can be spaced apart, such as by about 8 hours.

In certain embodiments, the third-medication component comprises phenobarbital. The phenobarbital can be administered to the patient orally in various amounts, typically in an amount of from about 1 to about 50, alternatively from about 5 to about 50, alternatively from about 10 to about 40, alternatively from about 30 to about 40, alternatively about 32.4, mg, or any range between the lowest and highest of these values. It is to be appreciated that the phenobarbital can be administered in other ways, if desired. In certain embodiments, the phenobarbital is administered in one or more doses. For example, the phenobarbital can be administered in about 32.4 mg doses multiple times, e.g. about 15 times. The doses can be spaced apart, such as by about 8 hours. In certain embodiments, the doses can be weaned, such as about every 8 hours for about three days, then about every 12 hours for about three more days, then about every 24 hours for about three days.

In certain embodiments, the third-medication component comprises ropinirole. The ropinirole can be administered to the patient orally in various amounts, typically in an amount of from about 0.1 to about 5, alternatively from about 0.1 to about 2.5, alternatively from about 0.1 to about 2, alternatively from about 0.25 to about 1.5, alternatively from about 0.25 to about 1, mg, or any range between the lowest and highest of these values. It is to be appreciated that the ropinirole can be administered in other ways, if desired.

In certain embodiments, the third-medication component comprises ondansetron. The ondansetron can be administered to the patient sublingually in various amounts, typically in an amount of from about 0.1 to about 25, alternatively from about 1 to about 20, alternatively from about 1 to about 10, alternatively from about 5 to about 10, alternatively about 8, mg, or any range between the lowest and highest of these values. It is to be appreciated that the ondansetron can be administered in other ways, if desired. In certain embodiments, the ondansetron is administered in one or more doses. For example, the ondansetron can be administered in about 8 mg doses multiple times, e.g. about four times. The doses can be spaced apart, such as by about 8 hours. It is also to be appreciated that the amounts described above for each of the medications can be used in different combinations with one another, provided the respective medication is included as/in the third-medication component.

Optionally, after terminating administration of the anesthetic component to the patient, a fourth-medication component is administered to the patient. Typically, the fourth-medication component is administered to the patient by the medical professional during the observation period of the patient, if necessary. The caregiver and/or patient may also administer the third-medication component, if necessary during or after the observation period. The fourth-medication component is useful for further mitigating drug withdrawal symptoms of the patient. The fourth-medication component can also be useful as a sleep aid for the patient. If utilized, the fourth-medication can be administered before, after, or simultaneous with the third-medication component. As such, the fourth-medication component can be utilized as necessary, but may not be required based on the particular patient.

The fourth-medication component can be administered to the patient by various methods understood in the art. For example, the fourth-medication component can be administered orally, intramuscularly, or combinations thereof. The specific administration technique can be based in part on the form of the medication(s). The fourth-medication component can be administered in various amounts, as described below.

Typically, the fourth-medication component comprises, consists essentially of, or consists of, gabapentin, dicyclomine, prochlorperazine, haloperidol, benzotropine, carisoprodol, diazepam, triazolam, zolpidem, or combinations thereof. For example, the fourth-medication component can comprise one or more of the aforementioned medications. In certain embodiments, the fourth-medication component comprises gabapentin, dicyclomine, prochlorperazine, haloperidol, benzotropine, carisoprodol, or combinations thereof. In other embodiments, the fourth-medication component comprises diazepam, triazolam, zolpidem, or combinations thereof.

These medications are available from a variety of sources. Those skilled in the art appreciate that these medications can be provided under a variety of brand names. For example, Neurontin® is a form of gabapentin, Bentyl® is a form of dicyclomine, Compazine® is a form of prochlorperazine, Haldol® is a form of haloperidol, Cogentin® is a form of benzotropine, Soma® is a form of carisoprodol, Valium® is a form of diazepam, Halcion® is a form of triazolam, and Ambien® is a form of zolpidem. The medications may also be available generically or under another brand name.

In certain embodiments, the fourth-medication component comprises gabapentin. The gabapentin can be administered to the patient orally in various amounts, typically in an amount of from about 1 to about 500, alternatively from about 100 to about 500, alternatively from about 200 to about 500, alternatively from about 200 to about 400, alternatively about 300, mg, or any range between the lowest and highest of these values. It is to be appreciated that the gabapentin can be administered in other ways, if desired. In certain embodiments, the gabapentin is administered in one or more doses. For example, the gabapentin can be administered in about 300 mg doses multiple times, e.g. about 30 times. The doses can be spaced apart, such as by about 8 hours.

In certain embodiments, the fourth-medication component comprises dicyclomine. The dicyclomine can be administered to the patient orally in various amounts, typically in an amount of from about 1 to about 50, alternatively from about 5 to about 40, alternatively from about 5 to about 30, alternatively from about 10 to about 30, alternatively about 20, mg, or any range between the lowest and highest of these values. It is to be appreciated that the dicyclomine can be administered in other ways, if desired. In certain embodiments, the dicyclomine is administered in one or more doses. For example, the dicyclomine can be administered in about 20 mg doses multiple times, e.g. about 20 times. The doses can be spaced apart, such as from about four to about six hours.

In certain embodiments, the fourth-medication component comprises prochlorperazine. The prochlorperazine can be administered to the patient orally and/or intramuscularly in various amounts, typically in an amount of from about 0.1 to about 25, alternatively from about 0.1 to about 20, alternatively from about 1 to about 15, alternatively from about 5 to about 10, mg, or any range between the lowest and highest of these values. It is to be appreciated that the prochlorperazine can be administered in other ways, if desired. In certain embodiments, the prochlorperazine is administered in one or more doses. For example, the prochlorperazine can be administered in about 5 to about 10 mg doses multiple times, e.g. about 4 to about 8 times. The doses can be spaced apart, such as by about 4 to about 6 hours. If employed, the total dosing of prochlorperazine should typically be about 40 mg or less within a span of 24 hours.

In certain embodiments, the fourth-medication component comprises haloperidol. The haloperidol can be administered to the patient orally and/or intramuscularly in various amounts, typically in an amount of from about 0.01 to about 10, alternatively from about 0.1 to about 10, alternatively from about 0.5 to about 5, alternatively from about 1 to about 5, alternatively from about 2 to about 5, mg, or any range between the lowest and highest of these values. It is to be appreciated that the haloperidol can be administered in other ways, if desired. In certain embodiments, the haloperidol is administered in one or more doses. For example, the haloperidol can be administered in about 2 to about 5 mg doses multiple times, as needed. The doses can be spaced apart, such as by about 1 to about 8 hours. If employed, the total dosing of haloperidol should typically be about 100 mg or less within a span of 24 hours.

In certain embodiments, the fourth-medication component comprises benzotropine. The benzotropine can be administered to the patient orally in various amounts, typically in an amount of from about 0.01 to about 10, alternatively from about 0.1 to about 10, alternatively from about 0.5 to about 5, alternatively from about 0.5 to about 1, alternatively about 1, mg, or any range between the lowest and highest of these values. It is to be appreciated that the benzotropine can be administered in other ways, if desired. In certain embodiments, the benzotropine is administered in one or more doses. For example, the benzotropine can be administered in about 1 mg doses multiple times, e.g. about 6 times. The doses can be spaced apart, such as by about 12 hours. Typically, if the fourth-medication comprises haloperidol, it further comprises at least benzotropine.

In certain embodiments, the fourth-medication component comprises carisoprodol. The carisoprodol can be administered to the patient orally in various amounts, typically in an amount of from about 1 to about 500, alternatively from about 100 to about 500, alternatively from about 250 to about 500, alternatively from about 300 to about 400, alternatively about 350, mg, or any range between the lowest and highest of these values. It is to be appreciated that the carisoprodol can be administered in other ways, if desired. In certain embodiments, the carisoprodol is administered in one or more doses. For example, the carisoprodol can be administered in about 350 mg doses multiple times, e.g. about 15 times. The doses can be spaced apart, such as from about 6 to about 8 hours.

In certain embodiments, the fourth-medication component comprises diazepam. The diazepam can be administered to the patient orally and/or intramuscularly in various amounts, typically in an amount of from about 0.1 to about 50, alternatively from about 1 to about 40, alternatively from about 5 to about 30, alternatively from about 5 to about 20, alternatively from about 10 to about 20, alternatively from about 5 to about 10, mg, or any range between the lowest and highest of these values. It is to be appreciated that the diazepam can be administered in other ways, if desired. In certain embodiments, the diazepam is administered in one or more doses. For example, the diazepam can be administered in about 5 to about 10 mg doses multiple times, e.g. 2 to 4 times a day. Dosage can depend on administration technique. For example, higher doses may be given intramuscularly and fewer times relative to oral dosing.

In certain embodiments, the fourth-medication component comprises triazolam. The triazolam can be administered to the patient orally in various amounts, typically in an amount of from about 0.01 to about 10, alternatively from about 0.05 to about 5, alternatively from about 0.05 to about 1, alternatively from about 0.05 to about 0.5, alternatively from about 0.25 to about 0.5, mg, or any range between the lowest and highest of these values.

In certain embodiments, the fourth-medication component comprises zolpidem. The zolpidem can be administered to the patient orally in various amounts, typically in an amount of from about 1 to about 500, alternatively from about 5 to about 250, alternatively from about 5 to about 100, alternatively from about 5 to about 50, alternatively about 10, mg, or any range between the lowest and highest of these values. It is to be appreciated that the zolpidem can be administered in other ways, if desired. If employed, the zolpidem can be taken about one hour prior to sleep. It is also to be appreciated that the amounts described above for each of the medications can be used in different combinations with one another, provided the respective medication is included as/in the fourth-medication component.

Typically, prior to final discharge of the patient, the patient is once again examined. Recommendations can be made for the patient, regarding further aftercare and/or treatment. Typically, an evaluation of the patient is performed by a physician and/or psychologist to develop and review a specific aftercare plan for the patient. The aftercare plan can be based on initial observations and/or examination of the patient, as described above.

The aftercare plan for the patient can include various aspects. The aftercare plan is useful for assisting the patient from once again becoming addicted to the drug(s), i.e., relapsing, after the detoxification procedure. As described above, the patient can be examined one or more times by a psychologist. The psychologist can design an individualized post procedure monitoring and support program for the patient.

If employed, the psychologist typically conducts a thorough psychological evaluation of the patient prior to the procedure. This evaluation can include an interview with the patient regarding history of drug use by the patient, living environment of the patient, life and work schedules of the patient, and/or emotional and psychological support resources available to the patient. This interview is conducted with the patient, and, optionally, with the caregiver(s) of the patient.

After consulting with the anesthesiologist utilized during detoxification of the patient, the psychologist generally designs an ongoing, comprehensive, one-on-one support program for the patient. The support program can last for various amounts of time, such as up to a year (or more). The support program can be conducted via various communication devices, such as through one or more websites, by internet messaging, by mobile messaging, and/or by telephone communications. In this way, the invention method not only treats the physical attributes of dependency, but also the psychological attributes of dependency to greatly reduce the likelihood of relapse. The patient's information is kept in confidence to aid in the recovery process.

Typically, the patient will have at least a weekly communication requirement with the psychologist by online survey, web conversation, telephone, or text. One on one meetings can also be arranged between the patient and a caregiver, such as a psychologist. Mobile applications be used to keep in contact, receive information, track progress, track triggers, etc. Telehealth technologies can be used, including both store-and-forward and real-time. After a degree of psychological stability has been attained, monitoring can become more lax, such as requiring monthly communication through the same channels. The channels can be available to the patient 24 hours a day, 7 days a week. The patient can have lifetime follow-up, if needed. Ongoing support for the patient is useful for preventing relapse of the patient. In this way, the method provides an all-inclusive treatment for the patient, which greatly decreases the chance for relapse.

In addition or alternate to the psychological support of the patient, the patient can also be given one or more medications during the aftercare period. For example, the patient can be given a naltrexone intramuscular injection, which is useful for prohibiting opiate effects. The injection can stay in the patient's body for an extended period of time, thus mitigating potential for relapse. Other subsequent naltrexone therapies can also be arranged to be provided for the patient when he/she returns home. For example, the patient may be given a naltrexone implant, which can last for many days, such as 60. These therapies may be arranged through a primary care physician of the patient's choice.

To induce compliance with provisions of the aftercare, the patient can be offered special incentives. For example, the patient can be offered a complimentary naltrexone implant, discounted subsequent naltrexone therapies, etc. Compliance of aftercare can be monitored via planned and/or random drug screening of the patient.

To reduce anxiety, nausea, and/or any joint or muscle discomfort typically experienced by conventional drugs users upon detoxification, a pulse stimulation (PSTIM) device may be prescribed and utilized by patient. The PSTIM device can help alleviate residual withdrawal symptoms for the patient and manage pain, if present.

Additional specific embodiments for care (or aftercare) of the patient are described immediately below. Further suitable steps for treatment and care of the patient are described in co-pending U.S. patent application Ser. No. 13/607,652 to James W. Carpenter et al. (the '652 application), the disclosure of which is incorporated herein by reference in its entirety to the extent it does not conflict with the general scope of the invention.

### Post-Procedure:

One or more post-procedural steps may also be utilized after the procedure itself. If employed, the post-procedural step(s) is/are typically conducted the same day of or soon after the procedure. The post-procedural step(s) can include one or more of the following steps described immediately below. It is to be appreciated that in certain embodiments, the invention procedure further includes one or more of the post-procedure steps. The post-procedure step(s) can be used alone or in combination with various pre-procedure step(s), e.g. as described above and/or in the '652 application introduced above. Likewise, the pre-procedure step(s) can be used alone or in combination with the post-procedure step(s).

Recommendations for continuing care can be discussed and reviewed with the patient and a caregiver of the patient. Evaluation or post-procedure recommendations for potentially necessary anxiolytics/benzodiazepine can be discussed with the patient and if needed, prescriptions can be written for the patient. The patient can be discharged alongside a caregiver with recommendations for continuing monthly intramuscular injections, multivitamin prescriptions, and/or recommendations for continuing care. Scheduled dates for subsequent monthly intramuscular injections are typically confirmed prior to discharge of the patient. It is believed that long term success rates are higher with continued treatment and psychological counseling of the patient. Each of the aforementioned steps can be used alone, or in various combinations with one another.

### “Continuing Care” of the Patient After the Procedure:

After the procedure, continued care and treatment of the patient can include one or more of the following:

Bio-psycho-social assessment of the patient:

Interpretive Summary;

Significant other/family education;

Medical and alternative support therapies for the patient; and,

An individualized aftercare plan for the patient.

Needs Assessment and various screening tests can be done at the discretion of an addiction specialist. The patient will generally have a repeat assessment in 12 months. Some of the aforementioned forms of care are described in greater detail below.

### Bio-Psycho-Social Assessment of the Patient Prior to the Procedure:

Prior to the procedure, professionals will engage in confidential one to one interviews with the patient and caregiver/significant other. This interview is designed to gain crucial information regarding addiction history to enable the professionals and physicians to design a customized recovery aftercare plan. Such an assessment of the patient can include one or more of the following:

Patient self-assessment;

Review of presenting problems;

Medical conditions of the patient;

Substance abuse history of the patient;

Employment status of the patient;

Family of origin assessment; and

Support systems for the patient.

### Interpretative Summary:

Professionals will summarize all of the assessment information, meet with the physician and together, they will plan a treatment and aftercare plan for the patient.

### Significant Other/Family Education:

Significant others and family members are greatly impacted by a loved one's addiction (i.e., the patient's addiction). A goal of the education program is generally to increase the understanding of the impact that the addictive behavior has on all significant relationships and to provide tools that will support the patient's return to drug-free lifestyle. These tools can include one or more of the following:

Addiction education:

Family roles education;

Coping strategies for changing unhealthy behavior patterns of the patient; and

Developing personal recovery plan for significant other/family members of the patient.

During the invention procedure, addiction therapists can meet with the patient's loved ones to provide them with these valuable tools.

### Medical and Alternative Support Therapies for the Patient:

Medical aftercare therapies can address any underlying conditions present at the time of the invention procedure. These therapies can help diminish the symptoms without the use of traditional addictive drugs.

### Individualized Aftercare Plan for the Patient After the Procedure:

The aftercare plan can include one or more of the following:

Weekly, individual mobile or internet sessions with a licensed addiction therapist enabling one to one contact with a therapist, scheduled within the patient's time constraints:

Weekly confidential and anonymous online group sessions, allowing the patient to participate in a therapist facilitated group with other recovering abusers (e.g. other patients);

Weekly questionnaires electronically transmitted from a therapist directly to the patient can keep the therapist apprised of the patient's recovery progress. If any recovery problems are indicated in the patient's responses, the therapist will recognize them and can initiate immediate relapse prevention interventions to assist the patient.

The following examples, illustrating the method of the present invention, are intended to illustrate and not to limit the invention.

### EXAMPLES

A day prior to the procedure, a patient chart is created and patient labels are prepared. The patient chart includes the patient's background information, including medical history, demographics, insurance information, etc. The patient's blood is drawn for lab evaluation and a pre-procedure EKG is done at this time. A stress test may be required if a physical examination of the patient so indicates.

A psychological and medical evaluation of the patient is conducted. The psychological evaluation is done by an identified psychologist and all medical information is reviewed. Payment is collected for the procedure and medications, and receipt is given. Post procedure instructions for family/friend caregiver are reviewed and conveyed. A pharmacy is contacted for medication preparation. A cursory physical examination of the patient is performed by a physician.

The day of the procedure, the patient is transferred to a procedure room via gurney. NPO status of the patient is verified. A 7 panel drug test is administered to the patient. If the patient is of childbearing age, a urine β-hCG test is done. The patient is then admitted. Consent to treatment is signed by the patient and the patient changes into a gown.

An IV line is started and medications of the first-medication component are administered. The medications can be given in various orders. The medications of the first-medication component, as well as their dosages and administration technique, are shown in TABLE I below. It is to be appreciated that each of the respective dosages can be increased or decreased, e.g. by ±100, 75, 50, 25, 10, 5, or 1, %, based for example on the diagnosis, weight, age, and/or sex of the patient. Other environmental factors may also impact the dosage levels. Each of these dosage levels/ranges is expressly contemplated.

TABLE I

| Medication | Dosage | | Admin. Tech. |
|---|---|---|---|
| gabapentin | 1200 | mg | oral |
| ropinirole | 0.5 | mg | oral |
| citric acid sodium citrate | 30 | ml | oral |
| famotidine | 20 | mg | piggy back |
| glycopyrrolate | 0.2 | mg | IV push |
| ondansetron | 4 | mg | IV push |
| dexamethasone | 10 | mg | IV push |
| midazolam | 2 | mg | IV push |

Vitals signs of the patient are taken and recorded. A paramedic inspects the patient for cuts, burns, bruises, sores, open wounds or scabs, records instances, and has the patient confirm observations with initials.

Before the procedure, the patient is supine on gurney and transported to a surgical suite. Monitoring devices are applied to the patient. These devices include a three lead EKG. In the event of pre-existing cardiac history, a five lead EKG may be utilized. The devices also include a continuous pulse oximeter (SpO2), as well as a non-invasive blood pressure monitor e.g. cuff. The patient is then prepped for induction of general anesthesia.

The patient is pre-oxygenated with an oxygen mask. IV induction of anesthesia is done based upon evaluation of airway and previously assessed medical conditions of the patient. Induction of anesthesia is generally achieved by IV infusion of propofol at a dose of 2.5-4.5 mg/kg. Once the airway has been established, a laryngeal mask airway (LMA) is placed and proper position is verified. Upon verification of optimal airway via the LMA, a continuous drip of propofol at doses of 300 mcg/kg/min is begun and adjusted to achieve adequate anesthesia of the patient.

Once general anesthesia of the patient is established, administration of the medications of the detoxification component is given via IV and intramuscular (IM) injections. Naloxone in a dosage of 1.2 mg is mixed in 50 ml of normal saline and administered to the patient via IV drip. Additional medications of the detoxification component, as well as their dosages and administration techniques, are shown in TABLE II below. The medications can be given in various orders.

TABLE II

| Medication | Dosage | | Admin. Tech. |
|---|---|---|---|
| ketorolac | 30 | mg | IV push |
| ketorolac | 30 | mg | IM injection |
| methacarbamol | 100 | mg | IV push |
| dolasetron | 12.5 | mg | IV push |
| trimethobenzamide | 100 | mg | IM injection |

Upon completion of naloxone drip, the following medications are given to the patient as the second-medication component. Medications of the second-medication component, as well as their dosages and administration techniques, are shown in TABLE III below. The medications can be given in various orders.

TABLE III

| Medication | Dosage | | Admin. Tech. |
|---|---|---|---|
| naltrexone | 1.5 | mg | IV push |
| naltrexone | 1.5 | mg | IV push¹ |
| clonidine | 0.15 | mg | IV push |
| clonidine | 0.15 | mg | IV push² |
| naltrexone | 500 | mg | IM injection |

^{1, 2}Second IV push is started 5 minutes after first IV push.

The IV propofol drip is discontinued to initiate emergence of the patient from anesthesia. The LMA is removed, and once adequate spontaneous respiration is established and vital signs are stable, the patient is transferred via gurney to a recovery area.

Patient post procedure evaluation begins, supervised by a physician, and includes re-application of the following monitors: continuous pulse oximeter (SpO2), cuff, and EKG. During this first recovery phase (Phase I-Recovery), continuous post-procedure monitoring occurs to observe for signs of withdrawal and vital sign stability of the patient. After 45-60 minutes period of stability, the patient is moved to Phase II-Recovery.

The patient continues to be monitored at regular intervals for vital sign stability and is continually assessed for withdrawal symptoms which may require medication for relief. This period of time lasts approximately 2-4 hours. Upon continued stabilization of the patient's vital signs, the IV line is removed. The monitors are also removed from the patient. The patient is then moved to Phase III-Recovery.

The patient is discharged to a pre-designated recovery location with caregiver. A paramedic resides with the patient until Phase III is complete. If there is no caregiver accompanying the patient, a personal nurse is assigned and attends to the patient, assisted by the paramedic for a 24-hour period. During this time, the paramedic and/or nurse examine the patient every four hours. The paramedic and/or nurse can be in contact with the physician at all times during this period. If necessary, the paramedic and/or nurse can administer the third-medication component, reestablish IV access if necessary, answer patient questions and provide aftercare comfort and support. Medications of the third-medication component, as well as their dosages and administration techniques, are shown in TABLE IV below. The medications can be given in various orders.

TABLE IV

| Medication | Dosage | | Admin. Tech. |
|---|---|---|---|
| clonidine | 0.1 | mg | oral³ |
| naltrexone | 50 | mg | oral⁴ |
| promethazine | 25 | mg | oral⁵ |
| phenobarbitol | 32.4 | mg | oral⁶ |
| ropinirole | 1 | mg | oral |
| ondansetron | 8 | mg | sublingually⁷ |

³Can be taken every 8 hours, up to 17 dosages
⁴Can be taken every 24 hours, up to 30 dosages.
⁵Can be takenevery 8 hours, up to 12 dosages.
⁶Can be taken as weaning dose every 8 hours for three days, then every twelve hours for three days, up to 15 dosages.
⁷Can be taken every 8 hours, up to four dosages.

After three days, the patient is then moved to Phase IV-Recovery. The patient is examined in consultation with supervising physician and/or paramedic. Upon a successful outcome, the patient is discharged to home with recommendations for further aftercare and treatment. An evaluation is performed by a physician and a psychologist to review specific aftercare plan designed for the patient based upon initial psychological evaluation(s).

The psychologist designs an individualized post procedure monitoring and support program for the patient. The evaluation includes an interview with the patient regarding history of drug use, living environment, life and work schedules, and emotional and psychological support available. The interview is conducted with the patient, and with any accompanying caregivers or family members. After consulting/conferring with the anesthesiologist, the psychologist designs an ongoing, comprehensive, one/one online and mobile support program for up to one year. This can be done through a website, mobile messaging, or telephone communications. The patient has a weekly communication requirement with the psychologist by online survey, web conversation, telephone, and/or text. After a degree of psychological stability has been attained, monitoring becomes monthly through the same channels.

As deemed necessary, the patient can have a naltrexone IM injection to prohibit opiate effects. Subsequent naltrexone therapies can be arranged to be provided for the patient when he/she returns home. If deemed necessary, the patient can be given a PSTIM device. This discreet pain relieving device can help to alleviate some residual withdrawal symptoms for the patient. The invention method has shown to have excellent success rates, such as an 80% success rate after a year. Said another way, 4 out of 5 patients generally remain drug free after a year relative to an industry average of less than 1 in 5 patients staying in compliance after a year.

The invention method, including various embodiments described herein, is generally available from Eagle Advancement Institute™ (EAI) of West Bloomfield Township, Mich. under the name Clarity™. Other names which may generally be associated with the invention method and EAI include Clarity Rapid Opiate Reversal™, Clarity Continuing Care™, IOOT™, and/or PSTIM™.

It is to be understood that the appended claims are not limited to express and particular compounds, compositions, or methods described in the detailed description, which may vary between particular embodiments which fall within the scope of the appended claims. With respect to any Markush groups relied upon herein for describing particular features or aspects of various embodiments, it is to be appreciated that different, special, and/or unexpected results may be obtained from each member of the respective Markush group independent from all other Markush members. Each member of a Markush group may be relied upon individually and or in combination and provides adequate support for specific embodiments within the scope of the appended claims.

It is also to be understood that any ranges and subranges relied upon in describing various embodiments of the present invention independently and collectively fall within the scope of the appended claims, and are understood to describe and contemplate all ranges including whole and/or fractional values therein, even if such values are not expressly written herein. One of skill in the art readily recognizes that the enumerated ranges and subranges sufficiently describe and enable various embodiments of the present invention, and such ranges and subranges may be further delineated into relevant halves, thirds, quarters, fifths, and so on. As just one example, a range “of from 0.1 to 0.9” may be further delineated into a lower third, i.e., from 0.1 to 0.3, a middle third, i.e., from 0.4 to 0.6, and an upper third, i.e., from 0.7 to 0.9, which individually and collectively are within the scope of the appended claims, and may be relied upon individually and/or collectively and provide adequate support for specific embodiments within the scope of the appended claims. In addition, with respect to the language which defines or modifies a range, such as “at least,” “greater than,” “less than,” “no more than,” and the like, it is to be understood that such language includes subranges and/or an upper or lower limit. As another example, a range of “at least 10” inherently includes a subrange of from at least 10 to 35, a subrange of from at least 10 to 25, a subrange of from 25 to 35, and so on, and each subrange may be relied upon individually and/or collectively and provides adequate support for specific embodiments within the scope of the appended claims. Finally, an individual number within a disclosed range may be relied upon and provides adequate support for specific embodiments within the scope of the appended claims. For example, a range “of from 1 to 9” includes various individual integers, such as 3, as well as individual numbers including a decimal point (or fraction), such as 4.1, which may be relied upon and provide adequate support for specific embodiments within the scope of the appended claims.

The present invention has been described herein in an illustrative manner, and it is to be understood that the terminology which has been used is intended to be in the nature of words of description rather than of limitation. Many modifications and variations of the present invention are possible in light of the above teachings. The present invention may be practiced otherwise than as specifically described within the scope of the appended claims. The subject matter of all combinations of independent and dependent claims, both single and multiple dependent, is herein expressly contemplated.

What is claimed is:

1. A method of detoxifying a patient addicted to at least one drug, said method comprising the steps of:
administering a first-medication component to the patient for mitigating drug withdrawal symptoms of the patient;
administering an anesthetic component to the patient for putting the patient in a sedated state;
administering a detoxification component to the patient for detoxifying the patient of the drug(s) while the patient is in the sedated state;
administering a second-medication component to the patient for further mitigating drug withdrawal symptoms of the patient while the patient is in the sedated state; and
terminating administration of the anesthetic component to the patient for reviving the patient from the sedated state;
wherein the first-medication component comprises gabapentin, ropinirole, citric acid sodium citrate, famotidine, glycopyrrolate, ondansetron, dexamethasone, midazolam, or combinations thereof.

2. A method as set forth in claim 1 wherein the patient is in the sedated state for a period of time less than about four hours.

3. A method as set forth in claim 1 wherein the first-medication component comprises gabapentin, ropinirole, citric acid sodium citrate, famotidine, glycopyrrolate, ondansetron, dexamethasone, and midazolam.

4. A method as set forth in claim 1 wherein the first-medication component is administered prior to the administration of the anesthetic component.

5. A method as set forth in claim 1 wherein the anesthetic component comprises propofol.

6. A method as set forth in claim 1 wherein the detoxification component comprises naloxone.

7. A method as set forth in claim 6 wherein the detoxification component further comprises ketorolac, methacarbamol, dolasetron, trimethobenzamide, or combinations thereof.

8. A method as set forth in claim 1 wherein the second-medication component comprises naltrexone, clonidine, or combinations thereof.

9. A method as set forth in claim 1 wherein the second-medication component comprises naltrexone.

10. A method as set forth in claim 9 wherein a first portion of the naltrexone is administered intravenously and a second portion of the naltrexone is administered intramuscularly.

11. A method as set forth in claim 1 further comprising the step of administering a third-medication component to the patient after terminating administration of the anesthetic component to the patient for further mitigating drug withdrawal symptoms of the patent.

12. A method as set forth in claim 11 wherein the third-medication component comprises clonidine, naltrexone, promethazine, phenobarbital, ropinirole, ondansetron, or combinations thereof.

13. A method as set forth in claim 11 further comprising the step of administering a fourth-medication component to the patient after administration of the third-medication component to the patient for further mitigating drug withdrawal symptoms of the patent.

14. A method as set forth in claim 13 wherein the fourth-medication component comprises gabapentin, dicyclomine, prochlorperazine, haloperidol, benzotropine, carisoprodol, diazepam, triazolam, zolpidem, or combinations thereof.

15. A method as set forth in claim 1 further comprising the step(s) of:
i) monitoring vital signs of the patient at least while the patient is in the sedated state; and/or
ii) ventilating the patient at least while the patient is in the sedated state.

16. A method as set forth in claim 1 further comprising the step(s) of:
i) gathering patient information prior to administration of the first-medication component to the patient and/or after terminating administration of the anesthetic component to the patient; and/or
ii) examining the patient before administration of the first-medication component to the patient and/or after terminating administration of the anesthetic component to the patient.

17. A method as set forth in claim 1 wherein the drug is an opiate.

18. A method of detoxifying a patient addicted to at least one opiate, said method comprising the steps of:
administering a first-medication component to the patient for mitigating opiate withdrawal symptoms of the patient;
administering an anesthetic component comprising propofol to the patient for putting the patient in a sedated state;
ventilating the patient at least while the patient is in the sedated state;
monitoring vital signs of the patient at least while the patient is in the sedated state;
administering a detoxification component comprising naloxone to the patient for detoxifying the patient of the opiate(s) while the patient is in the sedated state;
administering a second-medication component comprising naltrexone to the patient for further mitigating opiate withdrawal symptoms of the patient while the patient is in the sedated state;
terminating administration of the anesthetic component to the patient for reviving the patient from the sedated state;
administering a third-medication component to the patient after terminating administration of the anesthetic component to the patient for further mitigating opiate withdrawal symptoms of the patent; and
administering a fourth-medication component to the patient after administration of the third-medication component to the patient for further mitigating drug withdrawal symptoms of the patent.

19. A method as set forth in claim 18 wherein the first-medication component comprises gabapentin, ropinirole, citric acid sodium citrate, famotidine, glycopyrrolate, ondansetron, dexamethasone, midazolam, or combinations thereof.

20. A method as set forth in claim 19 further comprising the step(s) of:
i) gathering patient information before administration of the first-medication component to the patient and/or after terminating administration of the anesthetic component to the patient; and/or
ii) examining the patient before administration of the first-medication component to the patient and/or after terminating administration of the anesthetic component to the patient.
